(19) United States
(12) Patent Application Publication
(10) Pub. No.: US 2015/0073281 A1
(43) Pub. Date: Mar. 12, 2015

(54) GENERATING A FLOW-VOLUME LOOP FOR RESPIRATORY FUNCTION ASSESSMENT

(71) Applicant: XEROX CORPORATION, Norwalk, CT (US)

(72) Inventors: Lalit Keshav MESTHA, Fairport, NY (US); Eribaweimon SHILLA, Karnataka (IN); Edgar A. BERNAL, Webster, NY (US); Himanshu J. MADHU, Webster, NY (US)

(73) Assignee: XEROX CORPORATION, Norwalk, CT (US)

(21) Appl. No.: 14/023,654

(22) Filed: Sep. 11, 2013

(51) Int. Cl. A61B 5/087; A61B 5/08

(52) U.S. Cl. CPC A61B 5/0873; A61B 5/0806
USPC 600/473; 600/476

(57) ABSTRACT

What is disclosed is a system and method for generating a flow-volume loop for respiratory function assessment of a subject of interest in a non-contact, remote sensing environment. In one embodiment, a time-varying sequence of depth maps of a target region of a subject of interest being monitored for respiratory function is received. The depth maps are of that target region over a period of inspiration and expiration. The depth maps are processed to obtain a volume signal comprising a temporal sequence of instantaneous volumes. The time-varying volume signal is processed to obtain a flow-volume loop. Changes in a contour of the flow-volume loop are used to assess the subject's respiratory function. The teachings hereof find their uses in a wide array of medical applications where it is desired to monitor respiratory function of patients such as elderly patients, chronically ill patients with respiratory diseases and premature babies.

### TECHNICAL FIELD

The present invention is directed to systems and methods for generating a flow-volume loop for respiratory function assessment of a subject of interest in a non-contact, remote sensing environment.

### BACKGROUND

Monitoring respiratory events is of clinical importance in the early detection of potentially fatal conditions. Current technologies involve contact sensors the individual must wear which may lead to patient discomfort, dependency, loss of dignity, and further may fail due to a variety of reasons. Elderly patients and neonatal infants are even more likely to suffer adverse effects of such monitoring by contact sensors. Unobtrusive, non-contact methods are increasingly desirable for patient respiratory function assessment.

Accordingly, what is needed are systems and methods for generating a flow-volume loop for respiratory function assessment of a subject of interest in a non-contact, remote sensing environment.

### INCORPORATED REFERENCES

The following U.S. patents, U.S. patent applications, and Publications are incorporated herein in their entirety by reference.

“Processing A Video For Tidal Chest Volume Estimation”, U.S. patent application Ser. No. 13/486,637, by Bernal et al. which discloses a system and method for estimating tidal chest volume by analyzing distortions in reflections of structured illumination patterns captured in a video of a thoracic region of a subject of interest.

“Minute Ventilation Estimation Based On Depth Maps”, U.S. patent application Ser. No. 13/486,682, by Bernal et al. which discloses a system and method for estimating minute ventilation based on depth maps.

“Minute Ventilation Estimation Based On Chest Volume”, U.S. patent application Ser. No. 13/486,715, by Bernal et al. which discloses a system and method for estimating minute ventilation based on chest volume by analyzing distortions in reflections of structured illumination patterns captured in a video of a thoracic region of a subject of interest.

“Processing A Video For Respiration Rate Estimation”, U.S. patent application Ser. No. 13/529,648, by Bernal et al. which discloses a system and method for estimating a respiration rate for a subject of interest captured in a video containing a view of that subject's thoracic region.

“Respiratory Function Estimation From A 2D Monocular Video”, U.S. patent application Ser. No. 13/680,838, by Bernal et al. which discloses a system and method for processing a video acquired using an inexpensive 2D monocular video acquisition system to assess respiratory function of a subject of interest.

“Monitoring Respiration with a Thermal Imaging System”, U.S. patent application Ser. No. 13/103,406, by Xu et al. which discloses a thermal imaging system and method for capturing a video sequence of a subject of interest, and processing the captured images such that the subject's respiratory function can be monitored.

“Enabling Hybrid Video Capture Of A Scene Illuminated With Unstructured And Structured Illumination Sources”, U.S. patent application Ser. No. 13/533,605, by Xu et al. which discloses a system and method for enabling the capture of video of a scene illuminated with unstructured and structured illumination sources.

“Contemporaneously Reconstructing Images Captured Of A Scene Illuminated With Unstructured And Structured Illumination Sources”, U.S. patent application Ser. No. 13/533,678, by Xu et al. which discloses a system and method for reconstructing images captured of a scene being illuminated with unstructured and structured illumination sources.

“Respiratory Physiology: The Essentials”, John B. West, Lippincott Williams & Wilkins; 9^{th} Ed. (2011), ISBN-13: 978-1609136406.

### BRIEF SUMMARY

What is disclosed is a system and method for generating a flow-volume loop for respiratory function assessment of a subject of interest in a non-contact, remote sensing environment. In one embodiment, a time-varying sequence of depth maps of a target region of a subject of interest being monitored for respiratory function is received. The depth maps are of that target region over a period of inspiration and expiration. The depth maps are processed to obtain a volume signal comprising a temporal sequence of instantaneous volumes. The time-varying volume signal is processed to obtain a flow-volume loop. Changes in a contour of the flow-volume loop are used to assess respiratory function. The teachings hereof find their uses in a wide array of medical applications where it is desired to monitor respiratory function of patients such as elderly patients, chronically ill patients with respiratory diseases and premature babies in a non-contact, remote sensing environment.

Features and advantages of the above-described system and method will become apparent from the following detailed description and accompanying drawings.

### BRIEF DESCRIPTION OF THE DRAWINGS

The foregoing will be made apparent from the following detailed description taken in conjunction with the accompanying drawings:

FIG. 1 shows an anterior (front) view and a posterior (back) view of a subject of interest intended to be monitored for respiratory function assessment in accordance with the teachings hereof;

FIG. 2 plots the output of a spirometer of a person taking seven tidal breaths followed by maximal forced inspiration and maximal forced expiration and finishing with several tidal breaths;

FIG. 3 shows the subject of FIG. 1 having a plurality of reflective marks arrayed in a uniform grid on their anterior thoracic region and on their posterior thoracic region;

FIG. 4 shows the subject of FIG. 1 wearing a shirt with a uniform pattern of reflective dots arrayed in uniform grid with a one inch dot pitch along a horizontal and a vertical direction;

FIG. 5 shows a flow-volume loop of a normal subject;

FIG. 6 is a flow-volume loop of a subject with an obstructive pulmonary disease;

FIG. 7 is a flow-volume loop of a subject with a severe airway limitation on the expiratory phase indicating a chronic obstructive pulmonary disease;

FIG. 8 is a flow-volume loop of a subject with an intra-thoracic upper airway obstruction indicating limited airflow at the beginning of;

FIG. 9 is a flow-volume loop of a subject with an extra-thoracic upper airway obstruction indicating an airflow limitation during inspiration and expiration;

FIG. 10 illustrates one embodiment of an example image-based depth sensing device acquiring video images of the target region of the subject of FIG. 4 being monitored for respiratory function assessment;

FIG. 11 is a flow diagram which illustrates one example embodiment of the present method for obtaining a flow-volume loop for respiratory function assessment in a remote sensing environment;

FIG. 12 is a functional block diagram of an example networked system for implementing various aspects of the present method described with respect to the flow diagram of FIG. 11;

FIG. 13 plots volume data of a first test subject generated from the depth maps obtained from a video signal acquired using an image-based depth sensing device of a first subject over a period of few tidal breathing cycles, followed by a period of maximally forced breathing, followed by few tidal breathing cycles;

FIG. 14 plots flow data of a first test subject as estimated with the proposed method from the data of FIG. 13;

FIG. 15 is a flow-volume loop obtained by parametrically plotting the estimated flow data obtained during maximally forced breathing cycle of FIG. 14 as a function of the volume data of FIG. 13;

FIG. 16 is a flow-volume loop generated from the volume and flow curves corresponding to a first subject over a period of a few tidal breathing cycles;

FIG. 17 plots volume data of a second test subject;

FIG. 18 plots flow data of a second test subject;

FIG. 19 is a flow-volume loop generated from the volume and flow curves corresponding to a second subject over a period of maximally forced breathing;

FIG. 20 is a flow-volume loop generated from the volume and flow curves corresponding to a second subject over a period of few tidal breathing cycles;

FIGS. 21 and 22 compare the results obtained with our present system side-by-side with the respective ground-truth data obtained using a spirometer for the first test subject; and

FIGS. 23 and 24 compare the results obtained with our present system side-by-side with the respective ground-truth data obtained using a spirometer for the second test subject.

### DETAILED DESCRIPTION

What is disclosed is a system and method for generating a flow-volume loop for respiratory function assessment of a subject of interest in a non-contact, remote sensing environment.

### NON-LIMITING DEFINITIONS

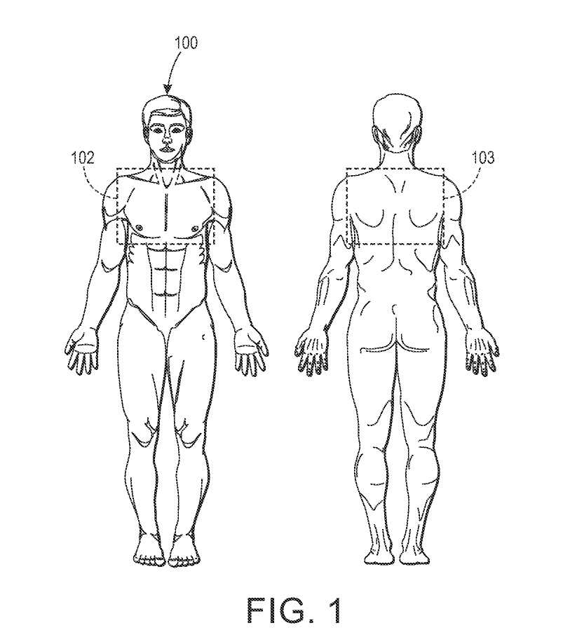

A “subject of interest” refers to a person being monitored for lung/respiratory function. It should be appreciated that the use of the terms “human”, “person”, or “patient” is not to be viewed as limiting the scope of the appended claims solely to humans. The teachings hereof apply equally to other subjects for which respiratory function is desired to be assessed. One example subject is shown in FIG. 1.

A “target region” of a subject, as used herein, refers to a subject's anterior thoracic region, a region of the subject's dorsal body, and/or a side view containing the subject's thoracic region. It should be appreciated that a target region can be any view of a region of the subject's body which can facilitate respiratory function assessment. FIG. 1 shows an anterior (frontal) view which outlines a target region 102 comprising the subject's anterior thoracic region. Target region 103 is of the subject's posterior thoracic region.

“Respiration”, as is normally understood, is a process of inhaling of air into lungs and exhaling air out of the lungs followed by a post-expiratory pause. Inhalation is an active process caused by a negative pressure having been induced in the chest cavity by the contraction of a relatively large muscle (often called the diaphragm) which changes pressure in the lungs by a forcible expansion of the lung's region where gas exchange takes place (i.e., alveolar cells). Exhalation is a passive process where air is expelled from the lungs by the natural elastic recoil of the stretched alveolar cells. The lining of alveolar cells has a surface-active phospholipoprotein complex which causes the lining to naturally contract back to a neutral state once the external force causing the cell to stretch has released. A post-expiratory pause occurs when there is an equalization of pressure between the lungs and the atmosphere. When the subject is at rest, the duration of the post-expiratory pause can be relatively long. The duration of the post-expiratory pause reduces with increased physical activity and may even fall to zero at very high rates of exertion.

“Forced inspiration” is when the subject forces the expansion of the thoracic cavity to bring more air into their lungs. During forced inhalation, external intercostal muscles and accessory muscles aid in expanding the thoracic cavity and bringing more air into the lungs. A maximally forced inspiratory breath is when the subject cannot bring any more air into their lungs. Total Lung Capacity (TLC) is the total volume of air in the lungs at maximal inspiration. TLC of an average adult human is about 6.0 liters of air. Restrictive pulmonary diseases are pulmonary fibrosis, pneumothorax, and Infant Respiratory Distress Syndrome decrease lung volume, whereas obstructive pulmonary diseases are asthma, bronchitis, and emphysema, and obstruct airflow.

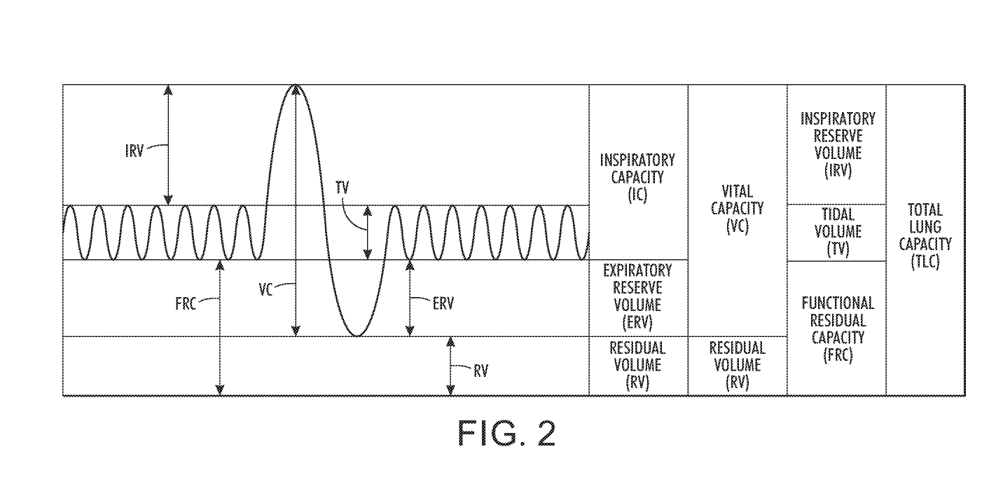

“Forced expiration” is when the subject forces the contraction of the thoracic cavity to expel air out of their lungs. During forced exhalation, expiratory muscles including abdominal and internal intercostal muscles, generate abdominal and thoracic pressure which helps expel air from the lungs. A maximally forced expiratory breath is when the subject cannot expel any more air from their lungs. FIG. 2 plots the output of a spirometer of a person taking seven tidal breaths followed by maximal forced inspiration and maximal forced expiration and finishing with several tidal breaths.

“Tidal chest volume” is the volume of air displaced by inspiration and expiration during normal breathing as opposed to heavy breathing due to exercise, for example.

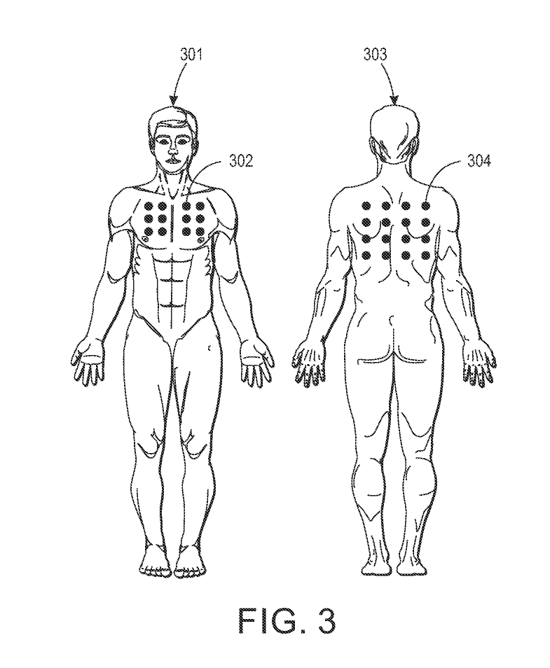

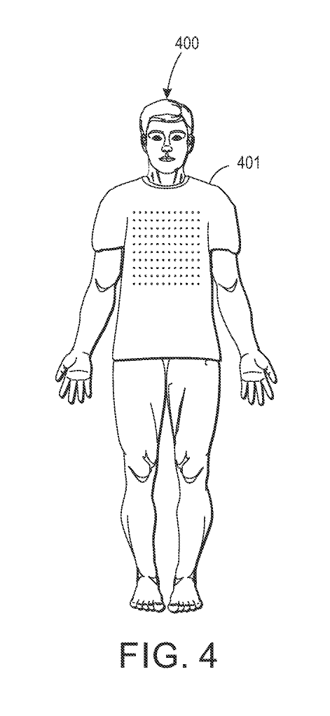

“Depth map sequence” is a reconstructed temporal sequence of 3D surface maps of a target region of a subject. There is a plurality of techniques known in the art for obtaining a depth map of a target region. For example, a depth map may be constructed based on the amount of deformation in a known pattern comprising, for instance, structured patterns of light projected onto the target region, textural characteristics present on the target region itself such as skin blemishes, scars, markings, and the like, which are detectable by a video camera's detector array. FIG. 3 shows a subject of interest 301 having a plurality of reflective marks arrayed in a uniform pattern 302 on an anterior thoracic region. Subject 303 is shown having a plurality of emissive marks such as LEDs arrayed in a uniform pattern 304 on their posterior thoracic region. The pattern may alternatively be an array of reflective or emissive marks imprinted or otherwise fixed to an item of clothing worn by the subject which emit or reflect a wavelength range detectable by sensors in a video camera's detector array. Reflective marks may be dots of reflective tape, reflective buttons, reflective fabric, or the like. Emissive marks may be LED illuminators sewn or fixed to the shirt. In FIG. 4, subject 400 is shown wearing shirt 401 with a uniform pattern of reflective dots arrayed in uniform grid with a 1 inch dot pitch along a horizontal and a vertical direction. It should be appreciated that the pattern may be a uniform grid, a non-uniform grid, a textured pattern, or a pseudo-random pattern so long as the pattern's spatial characteristics are known apriori. Higher-resolution patterns are preferable for reconstruction of higher resolution depth maps. Depth maps may be obtained from video images captured using an image-based depth sensing device such as a red green blue depth (RGBD) camera, a passive stereo camera, an infrared camera, an active stereo camera, an array of cameras, or a 2D monocular video camera. Depth maps may also be obtained from data acquired by non-image-based depth sensing devices such as a LADAR device, a LiDAR device, a photo wave device, or a time-of-flight measurement device as a depth measuring system. Depth maps can be obtained from data obtained by any of a wide variety of depth-capable sensing devices or 3D reconstruction techniques.

“Receiving depth maps” is intended to be widely construed and includes to download, upload, measure, estimate, obtain, or retrieve from a media such as a memory, hard drive, CDROM, DVD, measured from a depth-capable sensing device or the like. It should be appreciated that depth maps can be obtained using a camera to capture images of the subject while illuminated by a projected pattern of structured light, the camera being sensitive to a wavelength range of the structured light. The depth maps are then generated based upon a comparison of spatial characteristics of reflections introduced by a movement in the subject's chest cage to known spatial characteristics of the projected patterns, and using the characterized distortions at different locations to calculate the depth map for each image in the video. Such a method is taught in the above-incorporated reference by Bernal et al. Depth maps can be generated using distortions in patterned clothing worn by the subject as taught in the above-incorporated reference by Bernal et al. The embodiments herein are discussed with respect to the patterned clothing embodiment.

A “time-varying volume signal” is a signal comprising a temporal sequence of volumes at instantaneous intervals during inspiratory and expiratory breathing. Volume is obtained from processing each of the depth maps. In one embodiment, the depth map comprises a 3D hull defined by a set of 3D coordinates namely their horizontal, vertical and depth coordinates (x, y and z respectively). Points in the hull can be used to form a triangular tessellation of the target area. By definition of a tessellation, the triangles fill the whole surface and do not overlap. The coordinates of an anchor point at a given depth are computed. The anchor point can be located on a reference surface, for example, the surface on which the subject lies. The anchor point in conjunction with the depth map defines a 3D hull which has a volume. Alternatively, the coordinates of points on an anchor surface corresponding to the set of depths of a reference surface can be computed. The anchor surface in conjunction with the depth map also defines a 3D hull which has a volume. A volume can be computed for each 3D hull obtained from each depth map. A concatenation of all sequential volumes forms a volume signal comprising a temporal sequence of instantaneous volumes over the period of inspiration and expiration. This signal can be de-trended to remove low frequency variations and smoothed using a Fast Fourier Transform (FFT) or a filter. A peak detection algorithm can be further applied to the signal to help identify frequency components which, in turn, facilitate a determination of respiration rate.

It should be appreciated that, in environments where the patient is free to move around while being monitored for respiratory function, it may be necessary to build pose-dependent calibration functions specific to the device from which the depth maps are being derived. Data capture from different points of view can be performed and perspective-dependent volume signals derived. Processing from each point of view will lead to perspective-dependent or pose-dependent volume signals from which multiple calibration tables can be constructed. Calibration for various poses and perspectives intermediate to those tested can also be accomplished via interpolation.

A “flow-volume loop” is a plot or curve of a subject's inspiratory and expiratory air flow with respect to volume. The expiratory portion of a flow-volume loop is characterized by a rapid rise to the peak flow rate followed by a nearly linear fall in flow as the subject exhales toward residual volume. The inspiratory portion of the flow-volume loop is a relatively symmetrical, saddle-shaped curve. The flow rate at the midpoint of exhalation is normally approximately equivalent to the flow rate at the midpoint of inhalation. A flow-volume loop is obtained from processing the volume signal. In one embodiment, processing involves using a low-pass filter to filter the volume signal to obtain a filtered volume signal. A derivative with respect to time of the filtered volume signal is used to obtain a flow signal. A low-pass filter filters the flow signal to obtain a filtered flow signal. A flow-volume loop can be extracted from the filtered flow signal and the volume signal. Although filtering of the volume signal is often required to remove noise and to obtain a clean flow signal, it is not a requirement if the volume signal is acceptably noiseless to begin with.

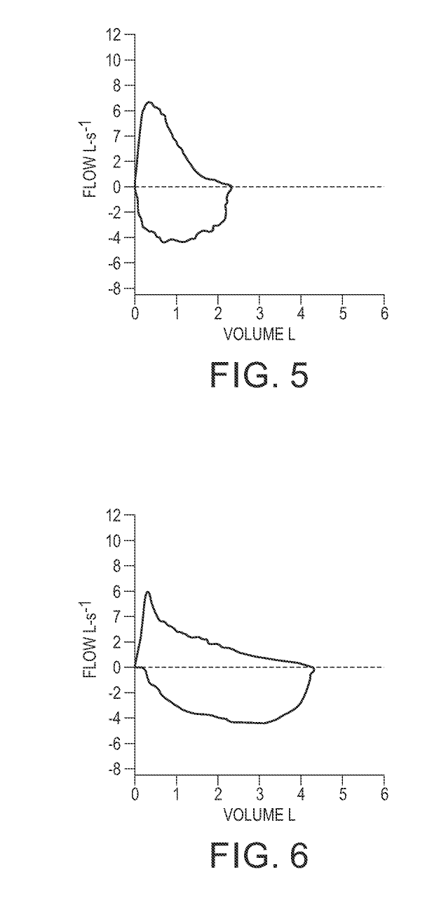

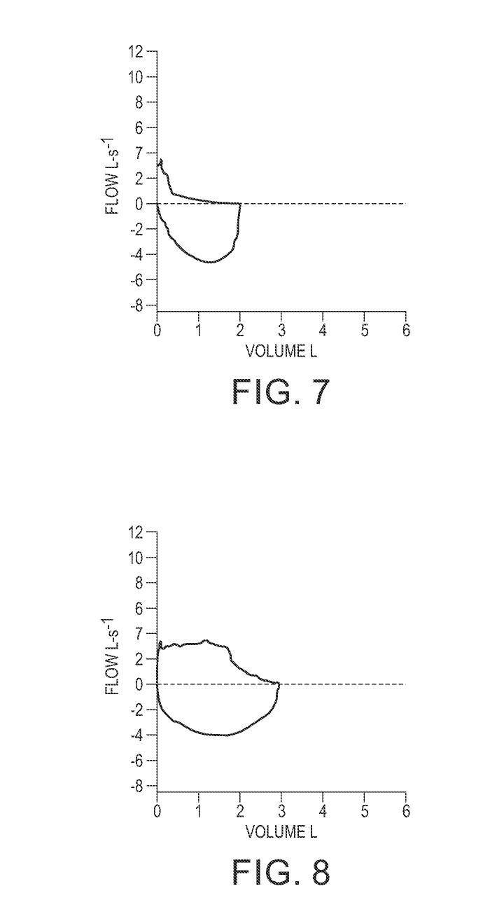

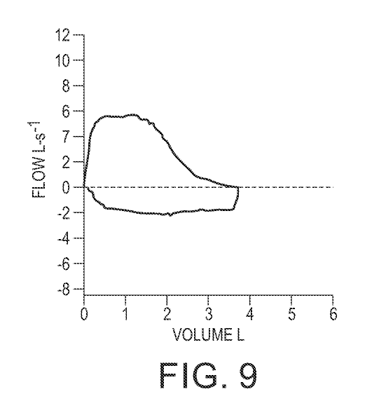

Changes in a contour of the flow-volume loop give a good indication of the state of the subject's respiratory function. FIGS. 5-9 show various flow-volume loops when the lungs are in various states. These figures are obtained from a contact-based spirometry. FIG. 5 shows a normal flow-volume curve with no airflow limitation. FIG. 6 shows a flow-volume loop with a flow limitation towards the end of expiration indicating an obstructive pulmonary disease such as asthma. FIG. 7 shows a flow-volume loop with a severe airway limitation on the expiratory phase indicating a chronic obstructive pulmonary disease. FIG. 8 is a flow-volume loop showing an intra-thoracic upper airway obstruction indicating a limited airflow on the y-axis at the beginning of expiration. FIG. 9 is a flow-volume loop showing an extra-thoracic upper airway obstruction indicating an airflow limitation on both the inspiration and expiration phase. Flow-volume loops can be used to assess certain forms of restrictive pulmonary diseases such as pulmonary fibrosis, pneumothorax, and Infant Respiratory Distress Syndrome as well as certain obstructive pulmonary diseases such asthma, bronchitis, and emphysema, and obstructed airflow.

A “remote sensing environment” refers to non-contact, non-invasive sensing, i.e., the sensing device does not physically contact the subject being sensed. The sensing device can be any distance away from the subject, for example, as close as less than an inch to as far as miles in the case of telemedicine. The environment may be any settings such as, for example, a hospital, ambulance, medical office, and the like.

### Example Depth Sensing System

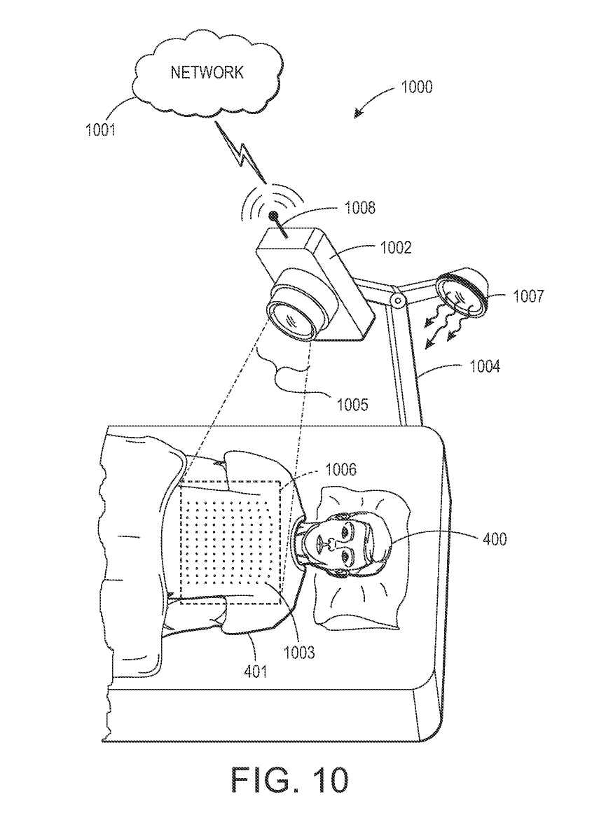

Reference is now being made to FIG. 10 which illustrates one embodiment of an example image-based depth sensing device acquiring video images of the target region of the subject of FIG. 4 being monitored for respiratory function assessment. In this embodiment, the image-based depth sensing device used to obtain video images of the subject's target region from which the time-varying sequence of depth maps is obtained can be, for example, a red green blue depth (RGBD) camera, an infrared depth camera, a passive stereo camera, an active stereo camera, or a 2D monocular video camera. In another embodiment where a non-image-based depth sensing device is used to acquire depth measurement data from which the time-varying sequence of depth maps is obtained can be, for example, a LADAR device, a LiDAR device, a photo wave device, or a time-of-flight measurement device.

Examination room 1000 has an example image-based depth sensing device 1002 to obtain video images of a subject 401 shown resting his/her head on a pillow while his/her body is partially covered by sheet. Subject 401 is being monitored for respiratory function assessment. Patient 401 is wearing a shirt 401 shown with a patterned array of reflective marks, individually at 1003. It is to be noted that clothing with patterned array of reflective marks is not needed when patterns are projected by the illumination source system. Video camera 1002 is rotatably fixed to support arm 1004 such that the camera's field of view 1005 can be directed by a technician onto target region 1006. Support arm 1004 is mounted on a set of wheels (not shown) so that video acquisition system 1002 can be moved from bed to bed and room to room. Although patient 400 is shown in a prone position lying in a bed, it should be appreciated that video of the target region 1006 can be captured while the subject is positioned in other supporting devices such as, for example, a chair or in a standing position. Video camera 1002 comprises imaging sensors arrayed on a detector grid. The sensors of the video camera are at least sensitive to a wavelength of illumination source system 1007 being reflected by the reflective marks 1003. The illumination source system may be any light wavelength that is detectable by sensors on the camera's detector array. The illumination sources may be manipulated as needed and may be invisible to the human visual system. The illumination source system may be arranged such that it may project invisible/visible patterns of light on the subject.

A central processor integral to the video camera 1002 and in communication with a memory (not shown) functions to execute machine readable program instructions which process the video to obtain the time-varying sequence of depth maps. The obtained sequence of depth maps may be wirelessly communicated via transmission element 1008 over network 1001 to a remote device operated by, for instance, a nurse, doctor, or technician for further processing, as needed, and for respiratory function assessment of patient 400. Alternatively, the captured video images are wirelessly communicated over network 1001 via antenna 1008 to a remote device such as a workstation where the transmitted video signal is processed to obtain the time-varying sequence of depth maps. The depth maps are, in turn, processed to obtain the time-varying volume signal and one or more flow-volume loops obtained from having processed the temporal sequence of instantaneous volumes. The resulting flow-volume loop(s) are then displayed on a monitor (not shown) either at a remote location or at the bedside of the patient such that the medical practitioner can visually examine changes in the contour of the flow-volume loop(s) to assess the subject's respiratory function. In other embodiments, the flow-volume loop is automatically examined by an artificial intelligence which analyzes the contours and outputs an alarm, notice, report, and the like, if a respiratory condition such as a pulmonary obstruction is identified. Camera system 1002 may further include wireless and wired elements and may be connected to a variety of devices via other means such as coaxial cable, radio frequency, Bluetooth, or any other manner for communicating video signals, data, and results. Network 1001 is shown as an amorphous cloud wherein data is transferred in the form of signals which may be, for example, electronic, electromagnetic, optical, light, or other signals. These signals may be communicated to a server which transmits and receives data by means of a wire, cable, fiber optic, phone line, cellular link, RF, satellite, or other medium or communications pathway or protocol. Techniques for placing devices in networked communication are well established. As such, further discussion as to specific networking techniques is omitted herein.

### Flow Diagram of One Embodiment

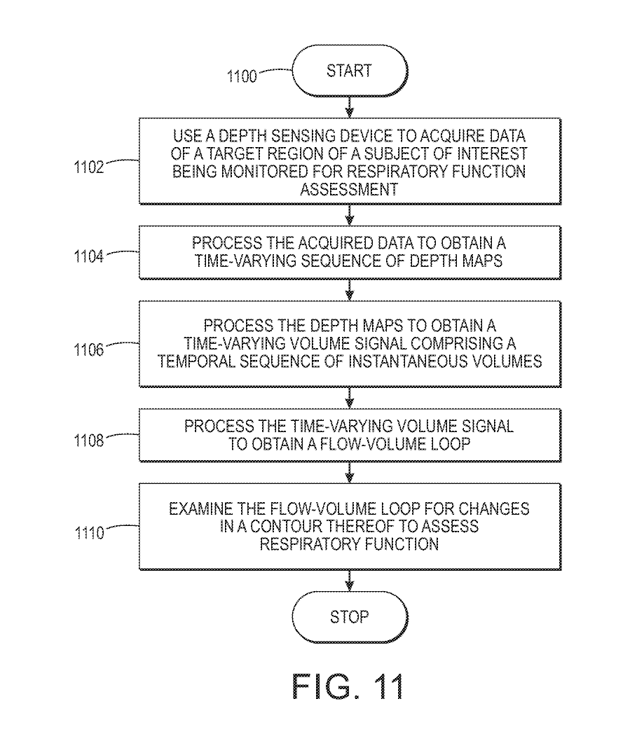

Reference is now being made to the flow diagram of FIG. 11 which illustrates one embodiment of the present method for obtaining a flow-volume loop for respiratory function assessment in a remote sensing environment. Flow processing begins at 1100 and immediately proceeds to step 1102.

At step 1102, use a depth sensing device to acquire data of a target region of a subject of interest being monitored for respiratory function assessment. The target region may be, for example, the subject's anterior thoracic region, a region of the subject's dorsal body, and a side view containing the subject's thoracic region. The depth sensing device may be an image-based depth sensing device or a non-image-based depth sensing device. Various example target regions are shown in FIG. 1.

At step 1104, process the acquired data to obtain a time-varying sequence of depths maps of the target region over a period of inspiration and expiration. The inspiration may be a maximal forced inspiration and the expiration a maximal forced expiration, or the inspiration and expiration are tidal breathing.

At step 1106, process the depth maps to obtain a time-varying volume signal comprising a temporal sequence of instantaneous volumes. If, in step 1104, the depth maps are obtained during a period wherein the subject's breathing is tidal breathing then the obtained time-varying volume signal is referred to herein as a tidal volume signal.

At step 1108, process the time-varying volume signal to obtain a flow-volume loop.

At step 1110, examine the flow-volume loop for changes in a contour thereof to assess the subject's respiratory function. Respiratory function assessment may involve a determination of a restrictive pulmonary disease, an obstructive pulmonary disease, and/or a localization of an airway obstruction. In this embodiment, further processing stops.

In another embodiment, the flow-volume loop is examined by an artificial intelligence algorithm to determine whether an alert condition exists. If so then an alert signal is automatically sent using, for example, transmissive element 1008 of FIG. 10. The alert signal may comprise, for example, a light blinking, an alarm or a message flashing on a monitor display. Such a notification can take the form of a text message sent to a cellphone of a medical practitioner such as a nurse, doctor or respiratory therapist. The notification alert may be a pre-recorded voice, text, or video message. Such an alert or notification can take any of a variety of forms and would depend on the particular environment wherein the teachings hereof find their intended uses. In another embodiment, a database is maintained for a given patient for enabling the medical practitioner to examine an evolution of that patient's pulmonary state. This can prove useful when examining a rate of progression of a particular lung disease.

It should be understood that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims. All or portions of the flow diagrams may be implemented partially or fully in hardware in conjunction with machine executable instructions.

### Example Networked System

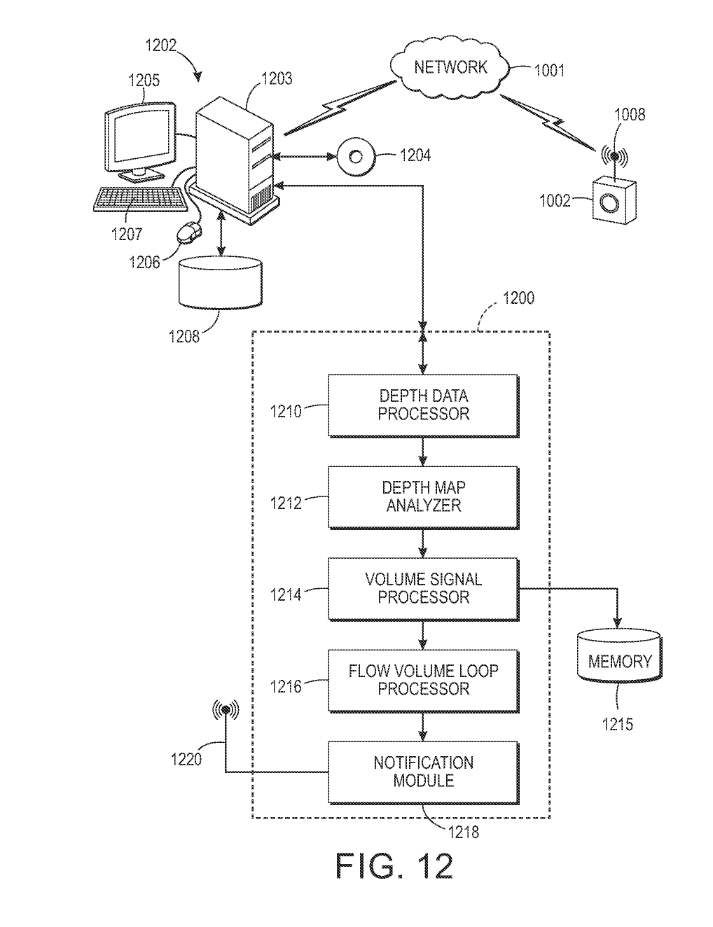

Reference is now being made to FIG. 12 which shows a functional block diagram of an example networked system for implementing various aspects of the present method described with respect to the flow diagram of FIG. 11. The system 1200 of FIG. 12 illustrates a plurality of modules, processors, and components placed in networked communication with a workstation 1202 wherein depth measurement data in the form of a video signal or depth values transmitted over network 1001 via transmissive element 1008 by depth sensing device 1002 are received for processing.

Workstation 1202 includes a hard drive (internal to computer housing 1203) which reads/writes to a computer readable media 1204 such as a floppy disk, optical disk, CD-ROM, DVD, magnetic tape, etc. Case 1203 houses a motherboard with a processor and memory, a communications link such as a network card, graphics card, and the like, and other software and hardware to perform the functionality of a computing device as is generally known in the arts. The workstation includes a graphical user interface which, in various embodiments, comprises display 1205 such as a CRT, LCD, touch screen, etc., a mouse 1206 and keyboard 1207. Information may be entered by a user of the present system using the graphical user interface. It should be appreciated that workstation 1202 has an operating system and other specialized software configured to display a wide variety of numeric values, text, scroll bars, pull-down menus with user selectable options, and the like, for entering, selecting, or modifying information displayed on display 1205. The embodiment shown is only illustrative. Although shown as a desktop computer, it should be appreciated that computer 1202 can be any of a laptop, mainframe, client/server, or a special purpose computer such as an ASIC, circuit board, dedicated processor, or the like. Any of the Information obtained from any of the modules of system 1200 including various characteristics of any of the depth sensors can be saved to database 1208.

Depth Data Processor 1210 processes the acquired data to obtain a time-varying sequence of depths maps of the target region over a period of inspiration and expiration. Depth Map Analyzer 1212 receives the time-varying sequence of depth maps from processor 1210 and proceeds to process the received depth maps to produce a time-varying volume signal comprising a temporal sequence of instantaneous volumes. Volume Signal Processor 1214 receives the time-varying volume signal and processes that volume signal to generate a flow-volume loop. Volume Signal Processor 1214 stores the data for flow-volume loops to Memory 1215. Flow Volume Loop Analyzer 1216 receives the generated flow-volume loop(s) and uses an artificial intelligence algorithm to examine the flow-volume loops for changes in a contour in order to perform a respiratory function assessment. Respiratory function assessment may involve a determination of a restrictive pulmonary disease, an obstructive pulmonary disease, and/or a localization of an airway obstruction. The artificial intelligence algorithm determines whether an alert condition exists which requires medical attention. If so then a signal is provided to Notification Module 1218 which sends an alert signal via antenna element 1220 to a nurse, doctor or respiratory therapist. Such an alert or notification can take any of a variety of forms. Notification Module may further communicate any of the values, data, diagrams, results generated by any of the modules of system 1200 to a remote device.

It should be understood that any of the modules and processing units of FIG. 12 are in communication with workstation 1202 via pathways (not shown) and may further be in communication with one or more remote devices over network 1001. Any of the modules may communicate with storage devices 1208 and memory 1215 via pathways shown and not shown and may store/retrieve data, parameter values, functions, records, and machine readable/executable program instructions required to perform their intended functions. Some or all of the functionality for any of the modules of the functional block diagram of FIG. 12 may be performed, in whole or in part, by components internal to workstation 1202 or by a special purpose computer system.

Various modules may designate one or more components which may, in turn, comprise software and/or hardware designed to perform the intended function. A plurality of modules may collectively perform a single function. Each module may have a specialized processor and memory capable of executing machine readable program instructions. A module may comprise a single piece of hardware such as an ASIC, electronic circuit, or special purpose processor. A plurality of modules may be executed by either a single special purpose computer system or a plurality of special purpose systems operating in parallel. Connections between modules include both physical and logical connections. Modules may further include one or more software/hardware components which may further comprise an operating system, drivers, device controllers, and other apparatuses some or all of which may be connected via a network. It is also contemplated that one or more aspects of the present method may be implemented on a dedicated computer system and may also be practiced in distributed computing environments where tasks are performed by remote devices that are linked through a network.

### Performance Results

Tests were conducted simultaneously on two male volunteers using (1) spirometry (for ground-truth) and (2) an image-based depth sensing device. Spirometry was done by hospital staff.

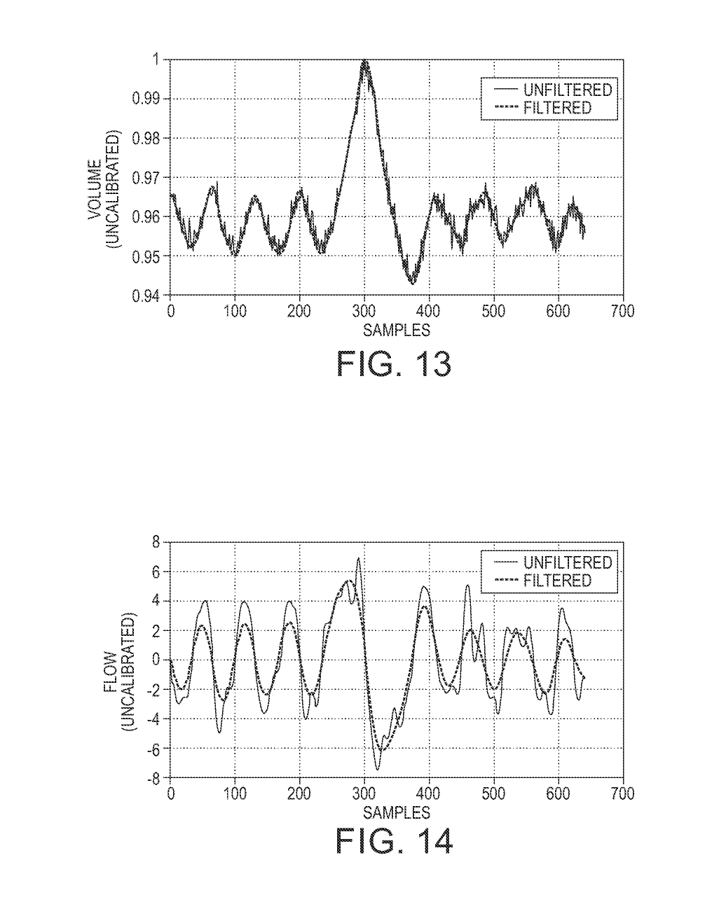

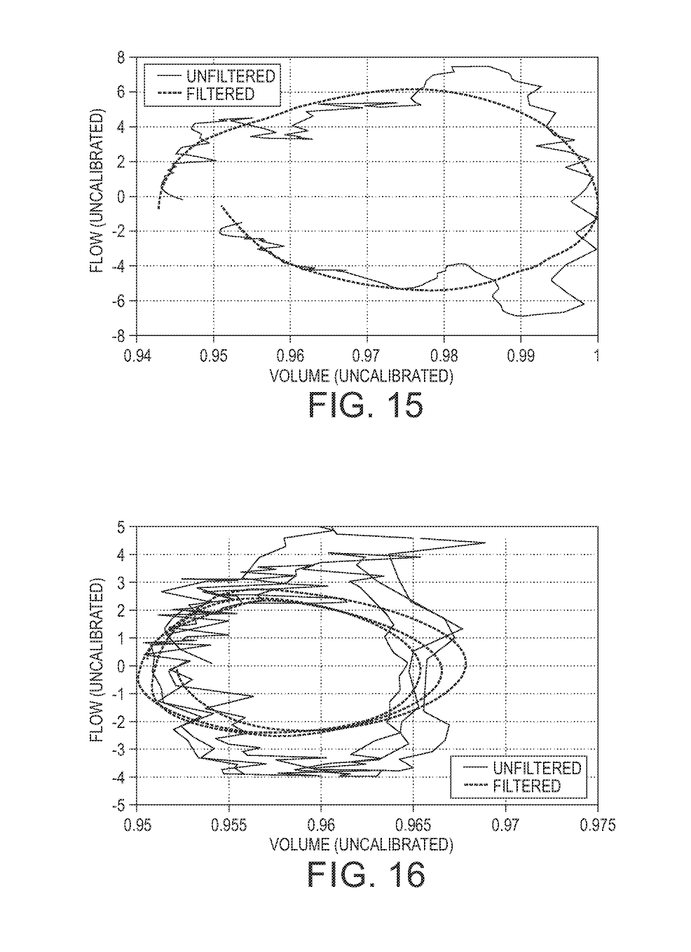

FIG. 13 plots volume data of a first test subject generated from the depth maps obtained from a video signal acquired using an image-based depth sensing device of a first subject over a period of few tidal breathing cycles, maximally forced breathing followed by few tidal breathing cycles. FIG. 14 plots flow data of a first test subject as estimated with the proposed method from the data from FIG. 13. FIG. 15 is a flow-volume loop obtained by parametrically plotting the estimated flow data obtained during maximally forced breathing cycle from FIG. 14 as a function of the generated volume data from FIG. 13. FIG. 15 used one cycle between sample 236 and sample 375 which corresponds to maximally forced breathing cycle. FIG. 16 is a flow-volume loop generated from the volume and flow curves corresponding to a first subject over a period of a few tidal breathing cycles. FIG. 16 used multiple cycles between sample 33 and sample 233.

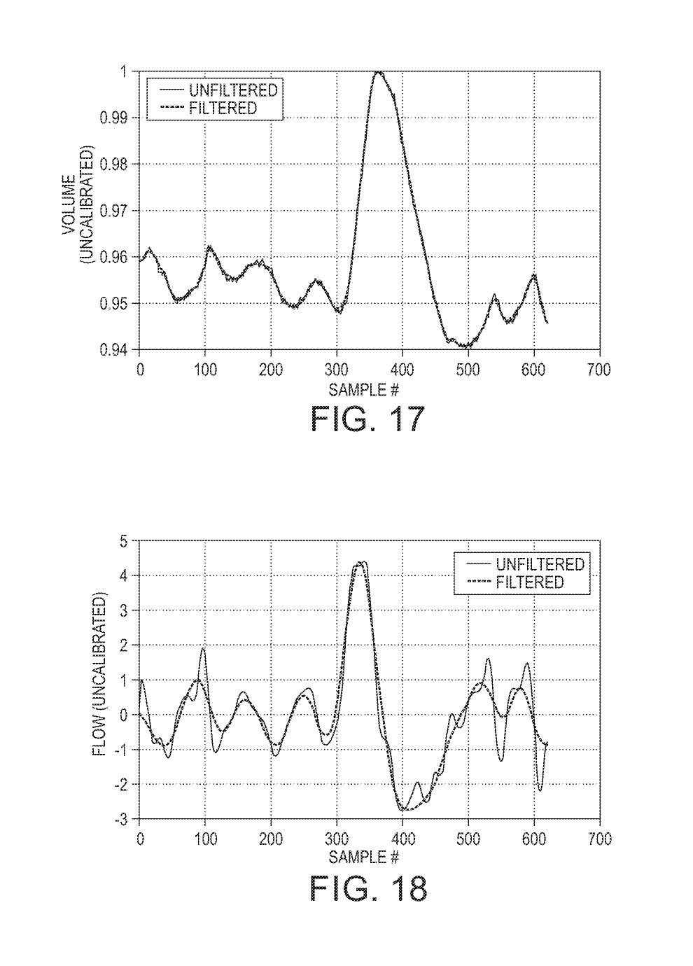

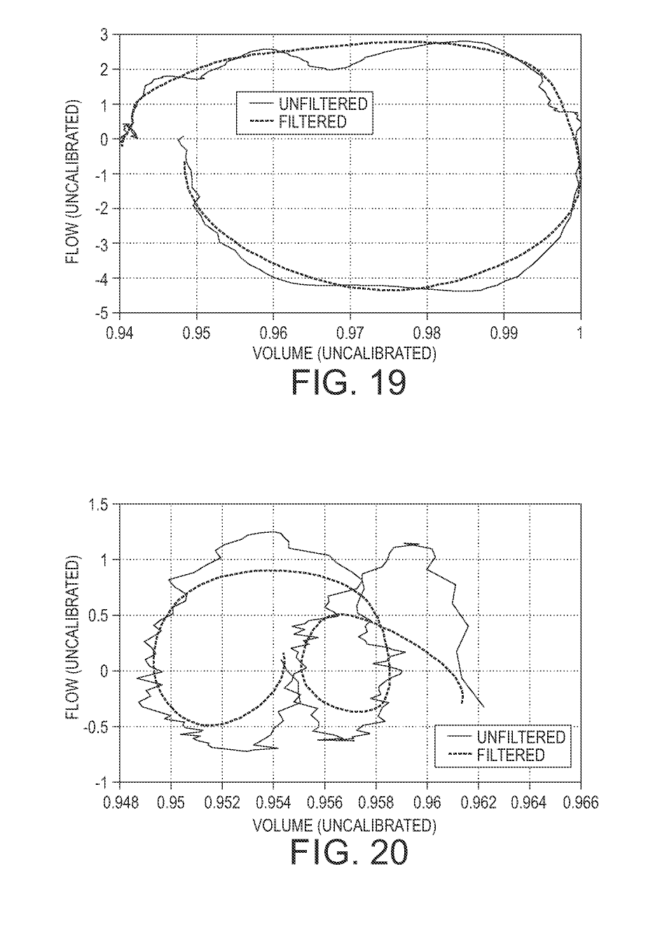

FIG. 17 plots volume data of a second test subject. FIG. 18 plots flow data of a second test subject. FIG. 19 is a flow-volume loop generated from the volume and flow curves corresponding to a second subject over a period of maximally forced breathing. FIG. 19 used one cycle between sample 302 and sample 495 from FIG. 18. FIG. 20 is a flow-volume loop generated from the volume and flow curves corresponding to a second subject over a period of few tidal breathing cycles. FIG. 20 used multiple cycles between sample 106 and sample 269 from FIG. 18.

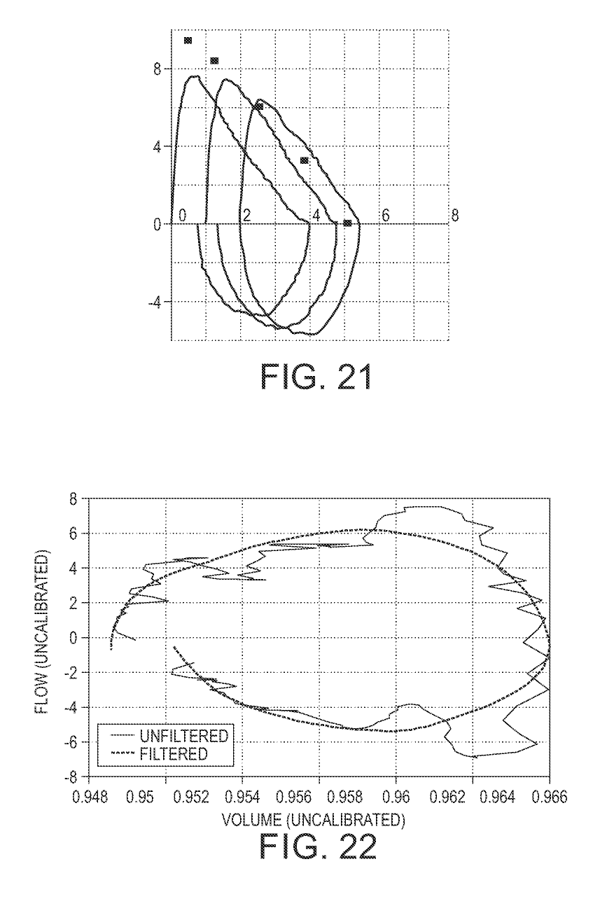

FIG. 21 and FIG. 22 compares the results obtained with our present system side-by-side with the respective ground-truth data obtained using a spirometer for the first subject.

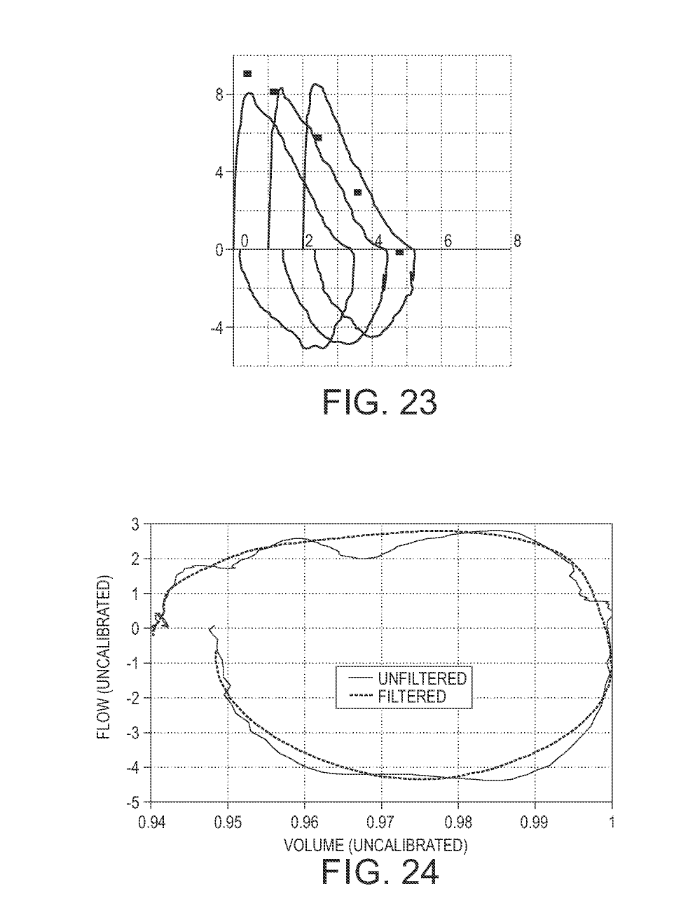

FIG. 23 and FIG. 24 compares the results obtained with our present system side-by-side with the respective ground-truth data obtained using a spirometer for the second subject.

As can be seen by an examination of the results hereof, the techniques disclosed herein generate flow-volume loops which substantially mirror that of the ground-truth data obtained using expensive and contact-based spirometry equipment.

### VARIOUS EMBODIMENTS

The teachings hereof can be implemented in hardware or software using any known or later developed systems, structures, devices, and/or software by those skilled in the applicable art without undue experimentation from the functional description provided herein with a general knowledge of the relevant arts.

One or more aspects of the methods described herein are intended to be incorporated in an article of manufacture, including one or more computer program products, having computer usable or machine readable media. The article of manufacture may be included on at least one storage device readable by a machine architecture embodying executable program instructions capable of performing the methodology and functionality described herein. Additionally, the article of manufacture may be included as part of a complete system or provided separately, either alone or as various components. It will be appreciated that various features and functions, or alternatives thereof, may be desirably combined into other different systems or applications. Presently unforeseen or unanticipated alternatives, modifications, variations, or improvements therein may become apparent and/or subsequently made by those skilled in the art, which are also intended to be encompassed with the scope of the following claims. Accordingly, the embodiments set forth above are considered to be illustrative and not limiting. Various changes to the above-described embodiments may be made without departing from the spirit and scope of the invention. The teachings of any printed publications including patents and patent applications, are each separately hereby incorporated by reference in their entirety.

What is claimed is:

1. A method for generating a flow-volume loop for respiratory function assessment in a remote sensing environment, the method comprising:
receiving a time-varying sequence of depth maps of a target region of a subject of interest being monitored for respiratory function, said depth maps being of said target region over a period of inspiration and expiration;
processing said depth maps to obtain a time-varying volume signal comprising a temporal sequence of instantaneous volumes; and
processing said time-varying volume signal to generate a flow-volume loop, characteristics in a contour of said flow-volume loop facilitating an assessment of said subject's respiratory function.

2. The method of claim 1, wherein said target region comprises one of: said subject's anterior thoracic region, a region of said subject's dorsal body, and a side view containing said subject's thoracic region.

3. The method of claim 1, wherein said depth maps are obtained from images captured using an image-based depth sensing device comprising any of: a red green blue depth (RGBD) camera, an infrared depth camera, a passive stereo camera, an array of cameras, an active stereo camera, and a 2D monocular video camera.

4. The method of claim 1, wherein said depth maps are obtained from data captured using a non-image-based depth sensing device comprising any of: a LADAR device, a LiDAR device, a photo wave device, and a time-of-flight measurement device.

5. The method of claim 1, wherein said depth maps are obtained from processing video images captured of said target region with patterned clothing using a video camera device comprising any of: a red green blue (RGB) camera, an infrared camera, a multispectral camera, and a hyperspectral camera.

6. The method of claim 1, wherein generating said flow-volume loop comprises:
taking a derivative of said volume signal to obtain a flow signal; and
extracting said flow-volume loop from said volume signal and said flow signal.

7. The method of claim 1, wherein generating said flow-volume loop comprises:
filtering, using a low-pass filter, said volume signal to obtain a filtered volume signal;
taking a derivative of said filtered volume signal to obtain a flow signal;
filtering, using a low-pass filter, said flow signal to obtain a filtered flow signal; and
extracting said flow-volume loop from said filtered volume signal and said filtered flow signal.

8. The method of claim 1, wherein said inspiration and expiration is tidal breathing and said volume signal corresponds to a tidal volume signal, said flow-volume loop being extracted from said tidal volume signal.

9. The method of claim 1, wherein said respiratory function assessment comprises using said flow-volume loop to facilitate a determination of any of: pulmonary disease, and a localization of an airway obstruction.

10. The method of claim 9, wherein said pulmonary disease is any of: pulmonary fibrosis, pneumothorax, Infant Respiratory Distress Syndrome, asthma, bronchitis, emphysema, and obstructed airflow.

11. The method of claim 1, further comprising storing said generated flow-volume loops over time for a given subject such that a rate of progression of said subject's respiratory function can be assessed.

12. The method of claim 1, further comprising compensating for an effect of a body motion of said subject by any of: an image-based image stabilization method, and a 3D surface stabilization method.

13. A system for generating a flow-volume loop for respiratory function assessment in a remote sensing environment, the system comprising:
a memory; and
a processor in communication with said memory, said processor executing machine readable program instructions for performing:
receiving a time-varying sequence of depth maps of a target region of a subject of interest being monitored for respiratory function, said depth maps being of said target region over a period of inspiration and expiration;
processing said depth maps to obtain a time-varying volume signal comprising a temporal sequence of instantaneous volumes;
processing said volume signal to generate a flow-volume loop; and
storing said flow-volume loop to said memory.

14. The system of claim 14, wherein said target region comprises one of: said subject's anterior thoracic region, a region of said subject's dorsal body, and a side view containing said subject's thoracic region.

15. The system of claim 14, wherein said depth maps are obtained from images captured using an image-based depth sensing device comprising any of: a red green blue depth (RGBD) camera, an infrared depth camera, a passive stereo camera, an array of cameras, an active stereo camera, and a 2D monocular video camera.

16. The system of claim 14, wherein said depth maps are obtained from data captured using a non-image-based depth sensing device comprising any of: a LADAR device, a LiDAR device, a photo wave device, and a time-of-flight measurement device.

17. The method of claim 14, wherein said depth maps are obtained from processing video images captured of said target region with patterned clothing using a video camera device comprising any of: a red green blue (RGB) camera, an infrared camera, a multispectral camera, and a hyperspectral camera.

18. The system of claim 14, wherein generating said flow-volume loop comprises:
taking a derivative of said volume signal to obtain a flow signal; and
extracting said flow-volume loop from said volume signal and said flow signal.

19. The system of claim 14, wherein generating said flow-volume loop comprises:
filtering, using a low-pass filter, said volume signal to obtain a filtered volume signal;
taking a derivative of said filtered volume signal to obtain a flow signal;
filtering, using a low-pass filter, said flow signal to obtain a filtered flow signal; and
extracting said flow-volume loop from said filtered flow signal and said volume signal.

20. The system of claim 14, wherein said inspiration and expiration is tidal breathing and said volume signal corresponds to a tidal volume signal, said flow-volume loop being extracted from said tidal volume signal.

21. The system of claim 14, wherein said respiratory function assessment comprises using said flow-volume loop to facilitate a determination of any of: pulmonary disease, and a localization of an airway obstruction.

22. The system of claim 21, wherein said pulmonary disease is any of: pulmonary fibrosis, pneumothorax, Infant Respiratory Distress Syndrome, asthma, bronchitis, emphysema, and obstructed airflow.

23. The system of claim 14, further comprising storing said generated flow-volume loops over time for a given subject such that a rate of progression of said subject's respiratory function can be assessed.

24. The system of claim 14, wherein said processor executes an artificial intelligence program to assess said subject' respiratory function.

25. The system of claim 14, further comprising compensating for an effect of a body motion of said subject by any of: an image-based image stabilization method, and a 3D surface stabilization method.
